(19) United States
(12) Patent Application Publication
(10) Pub. No.: US 2022/0319654 A1
(43) Pub. Date: Oct. 6, 2022

(54) SYSTEM AND METHOD OF EVALUATING A SUBJECT USING A WEARABLE SENSOR

(71) Applicant: KONINKLIJKE PHILIPS N.V., EINDHOVEN (NL)

(72) Inventors: Saman Parvaneh, Danvers, MA (US); Parastoo Alinia, Pullman, WA (US); Ali Akbar Ahmad Samadani, Somerville, MA (US)

(21) Appl. No.: 17/634,469

(22) PCT Filed: Aug. 19, 2020
(86) PCT No.: PCT/EP2020/073243

§ 371 Date: Feb. 10, 2022

Related U.S. Application Data
(60) Provisional application No. 62/889,308, filed on Aug. 20, 2019.

(51) Int. Cl. G16H 10/65; G06F 3/01; G16H 50/70

(52) U.S. Cl. CPC G16H 10/65; G06F 3/011; G16H 50/70

(57) ABSTRACT

A method of evaluating a subject using a wearable sensor includes collecting raw data at the sensor indicating movement and/or characteristics, determining a physical location of the sensor on the body of the subject, and determining whether the physical location matches a primary location on the subject corresponding to a location at which training data are collected for training pre-trained models. When the physical location matches the primary location, the physical activity and posture of the subject are determined using the raw data collected at the sensor, in accordance with a selected model. When the physical location does not match the primary location, the raw data is mapped from the physical location to the primary location using a machine-learning based algorithm to provide mapped data, and the physical activity and posture of the subject are determined using the mapped data, in accordance with the selected model.

### BACKGROUND

Human physical activities and postures recognition in commercial wearables, such as Apple Watch and Fitbit, for example, uses raw data collected and recorded at a specific location (e.g., the wrist) on the body of the subject. For example, wearable motion sensors, such as accelerometers and gyroscopes, are widely used to track human physical activities and postures. However, underlying algorithms or software models of the motion sensors are trained with respect to a specific location on the subject. Consequently, such motions sensors are not capable of maintaining high performance when worn on other locations on the body. For example, an Apple Watch has a model trained for a wrist location. This specification limits the users, since they have to adhere to a specific deployment protocols (e.g. wearing the sensors on the predefined body locations such as wrist).

Changing the location of a sensor may negatively impact its performance, and therefore may require retraining position specific models with new data and labels to create a complementary model. This process is time consuming and costly, and therefore is not practical in real-world settings.

Additionally, improvement of current human physical activities and postures recognition usually requires adding new data to increase the accessible database. Differences in recording protocols between different studies, such as changing the location of a sensor or even the sensor itself (e.g. accelerometer), limit application of the recorded data in one study (one location) for improvement of model created for another study (another location).

Therefore, an approach is needed to use a highly accurate model for evaluating human physical activities and postures, created for a primary location on the body of a subject at which data collection and physical activity/posture determination are especially accurate, regardless of whether the wearable sensor is worn at the primary location itself, or at some other secondary location. A secondary location is one different from the primary location, and has corresponding models the consistently yield less accurate results.

### SUMMARY

According to an aspect of the present disclosure, a method is provided for evaluating a subject using a wearable sensor worn on a body of the subject. The method includes collecting raw data at the sensor indicating movement and/or characteristics of the body; determining a physical location of the sensor on the body of the subject; and determining whether the physical location of the sensor matches a primary location on the body of the subject. The primary location corresponds to a location at which training data are previously collected for training pre-trained models, which are stored in a model database accessible to the sensor. Further according to the method, when the physical location of the sensor matches the primary location, at least one of posture and physical activity of the subject is determined using the raw data collected at the sensor, in accordance with a model selected from among the pre-trained models and retrieved from the model database. When the physical location of the source sensor does not match the primary location, the raw data is mapped from the physical location to the primary location using a machine-learning based algorithm to provide mapped data; and at least one of posture and physical activity of the subject is determined using the mapped data, in accordance with the selected model retrieved from the model database. The determined at least one of posture and physical activity of the subject is displayed on a display accessible to the sensor. Optionally, when the physical location of the source sensor does not match the primary location, the mapped data may be recorded in an augmented database, and the selected model may be retrained using the mapped data recorded in the augmented database, together with the training data.

According to another aspect of the present disclosure, a sensor device, wearable on a body of a subject, is provided for determining at least one of physical activity and posture of the subject. The sensor device includes a database that stores at least one pre-trained model previously trained using training data recorded from a primary location on the body; a memory that stores executable instructions including a sensor localization module, a physical activity and posture recognition module, and a sensor data mapping module; and a processor configured to execute the instructions retrieved from the memory. When executed by the processor, the instructions cause the processor to collect raw data indicating characteristics associated with the body in accordance with user instructions, to determine a location of the sensor device on the body of the subject in accordance with the sensor localization module, and to determine whether the determined location matches the primary location in accordance with the sensor localization module. When the determined location matches the primary location, the instructions further cause the processor to determine at least one of physical activity and posture of the subject, using the collected raw data and a pre-trained model selected from the at least one model stored in the database, in accordance with the physical activity and posture recognition module. When the determined location does not match the primary location, the instructions further cause the processor to map the raw data from the determined location to the primary location to provide mapped data, in accordance with the sensor data mapping module, and to determine at least one of physical activity and posture of the subject, using the mapped data, and a pre-trained model selected from the at least one model stored in the database, in accordance with the physical activity and posture recognition module. The sensor device further includes a display configured to display the at least one of physical activity and posture of the subject determined in accordance with the physical activity and posture recognition module.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a simplified block diagram of a system configured for evaluating a subject using a wearable sensor, in accordance with a representative embodiment.

FIG. 2 illustrates a simplified flow diagram of a process for evaluating a subject using a wearable sensor, in accordance with a representative embodiment.

FIG. 3 illustrates a simplified flow diagram showing a process for mapping raw data from a physical location to a primary location of a sensor using a machine-learning based algorithm, in accordance with a representative embodiment.

FIG. 4 illustrates a general computer system, on which a method of evaluating a subject using a wearable sensor may be implemented, in accordance with a representative embodiment.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a”, “an” and “the” are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Various embodiments of the present disclosure provide systems, methods, and apparatus for evaluating human physical activities and postures using a model created and previously trained using data from a primary (target) location on a subject, regardless of whether the actual location of a wearable sensor on the subject matches the primary location. That is, when the actual location of the sensor matches the primary location, the physical activities and postures are determined using raw data collected by the sensor applied to the pre-trained model. However, when the actual location of the sensor does not match the primary location (i.e., the actual location is a secondary location), the physical activities and postures are determined using mapped data applied to the pre-trained model, where the mapped data are obtained by mapping raw data collected by the sensor at the secondary location and mapped to the primary location using a machine-learning based algorithm. For example, according to various embodiments, the machine-learning based algorithm may map the sensor raw data recorded at the wrist (secondary location) of the subject to the chest (primary location) of the subject to be applied to the highly accurate pre-trained model. Also, in this case, the mapped data may be stored for use in retraining the pre-trained model to improve efficiency and accuracy of the pre-trained model. This enables, for example, the use of data recorded in one study in another study.

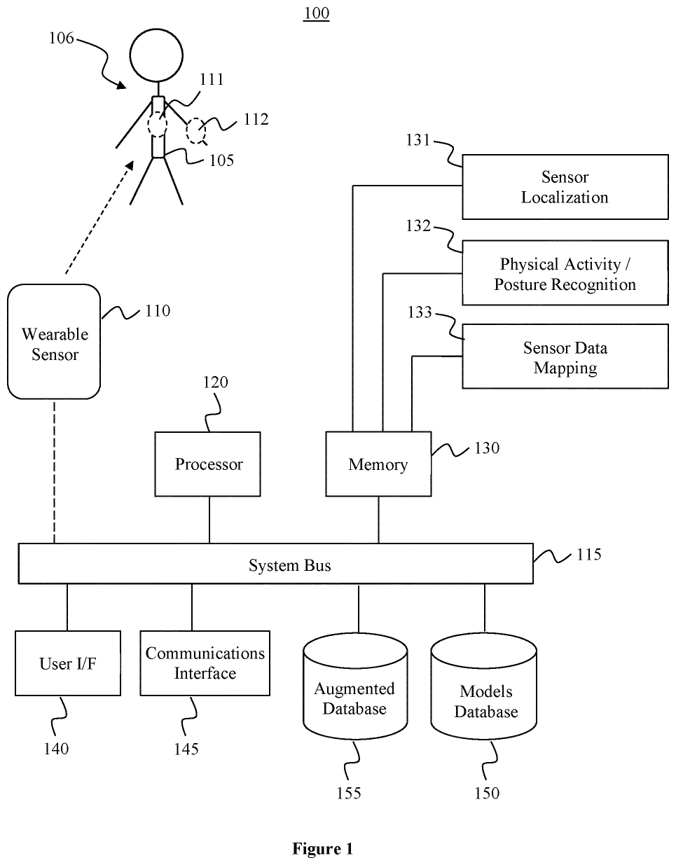

FIG. 1 illustrates a simplified block diagram of a system configured for evaluating a subject using a wearable sensor, in accordance with a representative embodiment. Referring to FIG. 1, a system 100 configured to execute the methods and/or models described herein for evaluating a subject 106 includes a wearable sensor 110 that is physically located on the body 105 of the subject 106. The evaluation includes identifying physical activity and posture of the subject 106. The wearable sensor 110 may be any device attachable to the body 105 for collecting raw data by monitoring one or more characteristics of the body 105 and/or ambient conditions. For example, the wearable sensor 110 may include one or more of an accelerometer, a gyroscope, a heart rate sensor, a thermometer, a barometer, and a microphone in order to provide various raw data related to characteristics associated with the subject 106 such as acceleration, physical movement, body position, heart rate, temperature, atmospheric pressure, and heart and lung sounds, for example, depending on the capabilities and location of the wearable sensor 110, as discussed below. The wearable sensor 110 may be a commercial wearable device, such as an Apple Watch or a Fitbit wearable on the wrist of the subject 106, or a Philips Lifeline wearable on or about the chest of the subject 106, for example. The raw data may be collected by the wearable sensor 110 itself, as well as by remote sensors (not shown) at various remote locations on the body 105 apart from the wearable sensor 110, the remote sensors being in communication with the wearable sensor 110 through wireless and/or wired connections. Notably, the data are collected at the wearable sensor, but could be saved/processed locally in the wearable sensor 110, or remotely in a cloud and/or a remote server.

The wearable sensor 110 may be attached to the body 105 at one of various locations, depending on its design. For example, the wearable sensor 110 may be attachable to the chest of the subject 106 at a primary location 111 or to the wrist of the subject 106 at a secondary location 112. The primary location 111 is generally better suited for collecting the raw data from the subject 106. For example, the wearable sensor 110 has access to additional information at the primary location 111, not available at the secondary location 112, such as heart and lung sounds, for evaluating the subject 106. Also, acceleration, physical movement and body position of the subject 106 may be more accurately and reliably detected by the wearable sensor 110 at the primary location 111, as opposed to having to determine these characteristics from more complex relative movements of extremities (e.g., wrist, ankle) to which the wearable sensor 110 may otherwise be attached, e.g., the secondary location 112. For example, the posture of the subject 106 being supine is more easily detected from the primary location 111, since the chest is horizontal when the body 105 is supine, whereas the extremities may be arranged at various orientations relative to the horizontal when the body 105 is supine.

The system 100 may further include, for example, a processor 120, memory 130, user interface 140, communications interface 145, models database 150, and augmented database 155 interconnected via at least one system bus 115. It is understood that FIG. 1 constitutes, in some respects, a simplified abstraction and that the actual organization of the components of the system 100 may be more complex than illustrated. Further, the wearable sensor 110 is shown connected to the system bus 115 by a dashed line, indicating that any combination of all or some of the processor 120, the memory 130, the user interface 140, the communications interface 145, the models database 150 and the augmented database 155 may be incorporated into the wearable sensor 110 itself, worn on the body 105 of the subject 106. For example, in an embodiment, the processor 120, the memory 130, the user interface 140 and the communications interface 145 may be located in the wearable sensor 110, enabling localized processing of raw data collected by the wearable sensor 110, while the models database 150 and the augmented database 155 may be located in a remote server(s) or cloud accessible to the wearable sensor 110 by a wireless network or wireless connection via the communications interface 145. Alternatively, in another embodiment, the communications interface 145 may be located in the wearable sensor 110, along with basic processing and user interfacing capability to enabling basic communications, such as sending out raw data and receiving processing results. Meanwhile, the processor 120, the memory 130, the models database 150 and the augmented database 155 may be located remote locations, such as in a remote server(s) and/or cloud, accessible to the wearable sensor 110 by a wireless network or wireless connection via the communications interface 145, enabling remote processing of the raw data collected by the wearable sensor 110. This embodiment may allow for more efficient processing and expanded storage. Other combinations of locations for the processor 120, the memory 130, the user interface 140, the communications interface 145, the models database 150 and the augmented database 155, including dividing of respective functionalities, between local and remote locations may be incorporated without departing from the scope of the present teachings.

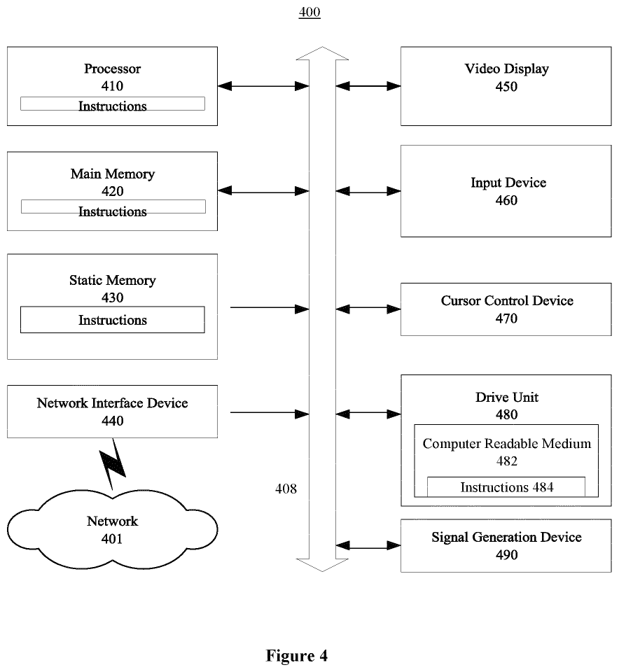

The processor 120 may be any hardware device capable of executing instructions stored in memory 130, the models database 150 and the augmented database 160, and otherwise processing raw data. As such, the processor 120 may include a microprocessor, field programmable gate arrays (FPGAs), application-specific integrated circuits (ASICs), or other similar devices, as discussed below with regard to processor 410 in illustrative computer system 400 of FIG. 4. The processor 120 may execute the instructions to implement part or all of methods described herein. Additionally, the processor 120 may be distributed among multiple devices, e.g., to accommodate methods necessarily implemented in a distributed manner that requires multiples sets of memory/processor combinations.

The memory 130 may include various memories such as, for example, cache or system memory. As such, the memory 130 may include static random-access memory (SRAM), dynamic RAM (DRAM), flash memory, read only memory (ROM), or other similar memory devices, as discussed below with regard to main memory 420 and/or static memory 430 in illustrative computer system 400 of FIG. 4. It will be apparent that, in embodiments where the processor includes one or more ASICs (or other processing devices) that implement one or more of the functions described herein in hardware, the software described as corresponding to such functionality in other embodiments may be omitted. That is, the memory 130 may store instructions for execution by the processor 120 and/or data upon with the processor 120 may operate.

The user interface 140 may include one or more devices for enabling communication with a user, such as the subject 106, a clinician, a technician, a doctor and/or other medical professional, for example. In various embodiments, the user interface 140 may be wholly or partially included on the wearable sensor 110, as mentioned above, for immediate access by the subject 106, and may include a display and keys, buttons and/or a touch pad or touch screen for receiving user commands. Alternatively, or in addition, the user interface 140 may include a command line interface or graphical user interface that may be presented to a remote terminal via the communication interface 145. Such remote terminal may include a display, a touch pad or touch screen, a mouse, and a keyboard for receiving user commands.

The communication interface 145 (e.g., network interface) may include one or more devices enabling communication by the wearable sensor 110 with other hardware devices. For example, the communication interface 145 may include a network interface card (NIC) configured to communicate according to the Ethernet protocol. Additionally, the communication interface 145 may implement a TCP/IP stack for communication according to the TCP/IP protocols, enabling wireless communications in accordance with various standards for local area networks, such as Bluetooth (e.g., IEEE 802.15) and Wi-Fi (e.g., IEEE 802.11), and/or wide area networks, for example. Various alternative or additional hardware or configurations for the communication interface 145 will be apparent.

Each of the models database 150 and the augmented database 155 may include one or more machine-readable storage media such as read-only memory (ROM), random-access memory (RAM), magnetic disk storage media, optical storage media, flash-memory devices, or similar storage media. In various embodiments, the models database 150 and the augmented database 155 may store instructions for execution by the processor 120 or data upon with the processor 120 may operate (alone or in conjunction with the memory 130). For example, the models database 150 may store one or more pre-trained models for determining physical activities and/or postures of a subject (e.g., such as the subject 106). Generally, each of the models is trained based on training data acquired by a sensor mounted on the chest of a training subject, or a simulation of the same, since data from a chest sensor is more accurate and tends to enable high performance. That is, the training data would be recorded at a training location corresponding to the primary location 111 on the body 105. Of course, the training data may be acquired from a training location other than the chest, without departing from the scope of the present teachings, in which case the primary location of the wearable sensor 110 for subsequent determination of physical activities and postures of a subject would correspond to the location from which the training data is acquired.

Notably, model training may be done one time in a computer using, for example, Windows, Mac, or Linux. Information of a model (e.g., weights of a neural network) is saved along with evaluation code. New data are fed to the evaluation code and the code uses the saved model for activity/posture recognition.

The training data may be collected from the actual subject 106, or from a test subject, representative of the universe of subjects, for the purpose of training the models. Alternatively or additionally, the training data may be simulated. As mentioned above, the training data is collected from a location corresponding to the primary location 111 since information regarding movement and positioning of the subject 106 tends to be more accurate as compared to information obtained from a secondary location (e.g., such as the secondary location 112). Also, more information is available at the primary location 111, such as heart and lung sounds, chest movement, body position and orientation, core temperature, and the like, which is not otherwise available from the secondary location 112. Each of the pre-trained models may include processor executable instructions for determining physical activities and postures based on the training data as applied to the model. The models may be recurrent neural network models with Long Short-Term Memory (LSTM) units, for example.

In accordance with certain representative embodiments, the models are trained and their performance is verified through splitting the training data into at least train and test sets, where the train set is used to train a model, and the test set is used to test the performance of the trained model. Different subsampling of the training data may be used to create train and test sets. For example, a hold-out set accounting for 30% of the training data can be set aside as a test set, and the remaining 70% of the training data may be used as a train set. The process of data splitting, model training and model verifying may be repeated for a number of times (e.g., 100) to collect performance statistics for a model. The performance statistics may include, but are not limited to accuracy, sensitivity, specificity, and precision, for example. When the model has hyper-parameters, for example the architecture of neural network classifiers including number of layers and activation functions, a part of the training data may be used as a validation set to tune these hyper-parameters.

The augmented database 155 collects and stores data that has been mapped from the secondary location 112 to the primary location 111 in order to determine physical activities and postures of the subject 106 using a selected model from the pre-trained models stored in the models database 150. For example, the mapped data may be the output of sensor data mapping module 133, discussed below. The process of mapping data between sensors using a selected model is discussed below. The selected model may then be retrained using the mapped data from the augmented database 155. The retrained (or augmented) selected model may then be stored again in the models database 150, where it is available for another study. Re-training the models stored in the models database 150 may improve future performance of the system 100. The memory 130 may include various modules, each of which comprises a set of related processor executable instructions corresponding to a particular function of the system 100. For example, in the depicted embodiment, the memory 130 includes sensor localization module 131, physical activity/posture recognition module 132, and sensor data mapping module 133. The sensor localization module 131 includes processor executable instructions for determining the physical location of the wearable sensor 110 on the body 105 of the subject 106, and for determining changes to the physical location of the wearable sensor 110, using raw data collected by the wearable sensor 110. In the depicted example, the sensor localization module 131 enables determination of whether the wearable sensor 110 is being worn at the primary location 111 or the secondary location 112, although other locations may be determined, without departing from the scope of the present teachings. The raw data collected by the wearable sensor 110, and trends or trend changes of the raw data, such as barometric pressure, may be used as potential features in a classifier model that identifies any changes in the location of the wearable sensor 110, for example. After detecting possible changes in the location of the wearable sensor 110, the sensor localization module 131 may detect the new location of the wearable sensor 110 on the body 105 with another model, such as another classifier model, which receives input data (e.g., barometric pressure) and provides a class (e.g., wrist, ankle, or chest) in the output.

Another way of detecting the location of the wearable sensor 110 is with a microphone, mentioned above. That is, when audio data providing the heart sound and/or lung sound are captured by the microphone, it indicates that the wearable sensor 110 is worn on the chest (e.g., primary location 111). Otherwise, the wearable sensor 110 may be on the wrist (e.g., secondary location 112), the ankle or other location remote from the heart and lungs of the subject 106. Also, for example, the location of the wearable sensor 110 may be detected by receiving acceleration data from an accelerometer and/or a gyroscope on the wearable sensor 110 that indicate movement of the sensor relative to the body 105 of the subject 106, which would indicate that the location of the wearable sensor 110 is on an extremity. The physical activity/posture recognition module 132 includes processor executable instructions for detecting physical activities and postures of the subject 106, based on an assumption that the wearable sensor 110 is at the primary location 111 on the body 105, using a selected pre-trained model retrieved from the models database 150, discussed above. The pre-trained model is selected, for example, by the user (e.g., the subject 106 or other person) through the user interface 140, or may be selected automatically based on information from sensor localization as described above. The sensor data mapping module 133 includes processor executable instructions for mapping the raw data from the secondary location 112 (or other secondary location) on the body 105 to the primary location 111 whenever the actual location of the wearable sensor 110, as determined by the sensor localization module 131, is at the secondary location 112 instead of the primary location 111. So ultimately, the physical activity/posture recognition module 132 detects the physical activities and postures of the subject 106 as though the wearable sensor 110 were at the primary location 111, either by processing the raw data directly when the wearable sensor 110 is actually located at the primary location 111 or by processing the mapped data from the sensor data mapping module 133 when the wearable sensor 110 is located at the secondary location 112. One or more of the detected physical activities and postures may be output by the physical activity/posture recognition module 132 via the user interface 140. For example, the user interface 140 may be connected to a display on which the one or more detected physical activities and postures may be displayed. Additional outputs may include warning devices or signals that correspond to various detected physical activities and postures. For example, an audible alarm or visual indicator may be triggered when the output indicates that a detected physical activity or change in postures is consistent with a fall by the subject. In an embodiment, the sensor data mapping module 133 maps the raw data from the secondary location 112, which is the determined physical location of the wearable sensor 110, to the primary location 111 using a machine-learning based algorithm to provide the mapped data. This may be done while the subject 106 continues to wear the wearable sensor 110 at the secondary location 112. For example, the sensor data mapping module 133 may use a recurrent neural network with long short-term memory (LSTM) units that map a source time series to a target time series, for example. Since a corresponding axis between two different sensor locations, e.g., the primary and secondary sensor locations 111 and 112, may change due to differences in the device (e.g., an accelerometer in the wearable sensor 110), utilized sensors, or how the subject 106 is wearing the wearable sensor 110, using methods such as correlation to find corresponding axis in the two sensor locations will improve the performance. The alignment of local coordinate frames of the sensors may also be done based on the angular velocities derived from or captured by the accelerometers, for example. Kinematic body models may also be used to transfer coordinate frame of one sensor to another based on the kinematic links and joins between the two body parts where the sensors are attached. This illustrative approach is generally known to one of ordinary skill in the art in robotics, and multi-body mechanical systems, where each body moves independantly of another and its motion can be described within its own local frame or the local frame of another body.

It will be apparent that information described as being stored in the models database 150 and/or the augmented database 155 may be additionally or alternatively stored in the memory 130. That is, although depicted separately, the models database 150 and the augmented database 155 may be included in the same physical database or in the memory 130. In this respect, the memory 130 may also be considered to constitute a “storage device” and the models database 150 and/or the augmented database 155 may be considered “memory.” Various other arrangements will be apparent. Further, the memory 130, the models database 150 and the augmented database 155 each may be considered to be “non-transitory machine-readable media.” As used herein, the term “non-transitory” will be understood to exclude transitory signals but to include all forms of storage, including both volatile and non-volatile memories.

While the system 100 is shown as including one of each described component, the various components may be duplicated in various embodiments. For example, the processor 120 may include multiple microprocessors that are configured to independently execute the methods described herein or are configured to perform steps or subroutines of the methods described herein such that the multiple processors cooperate to achieve the functionality described herein. Further, where the computer system 400 is implemented in a cloud computing system, the various hardware components may belong to separate physical systems. For example, the processor 120 may include a first processor in a first server and a second processor in a second server.

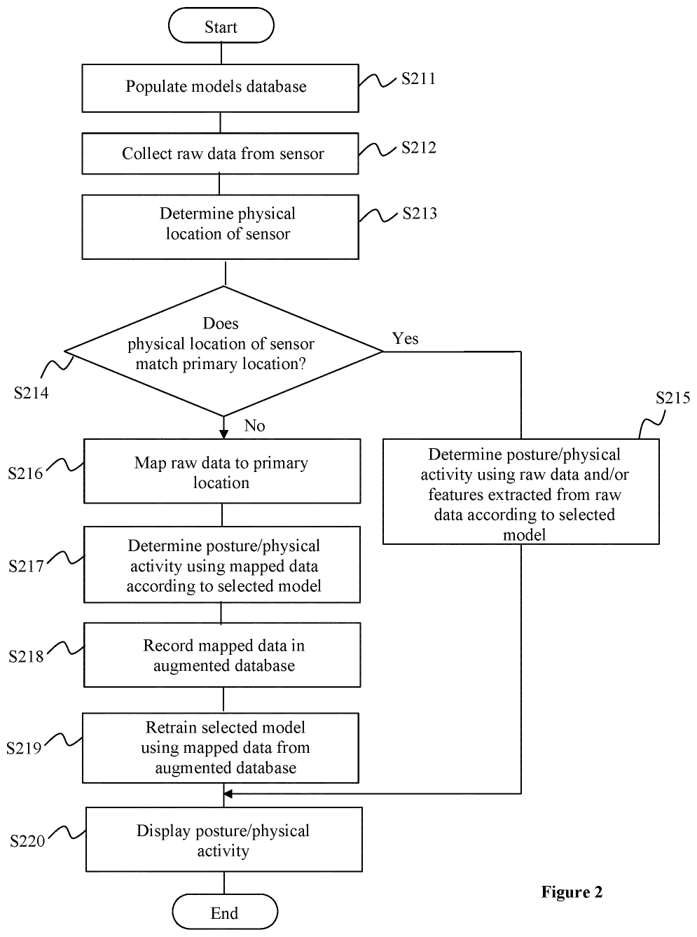

FIG. 2 is a flowchart showing a process for evaluating a subject using a wearable sensor on a body of a subject, according to a representative embodiment, that may be executed by the various systems described herein. The sensor may be worn at various different locations on the body, although one of the possible locations (primary location) provides better raw data to the sensor and/or better analysis by the sensor than other possible location(s) (secondary location(s)).

Referring to FIG. 2, all or a portion of the steps may be performed by the processor 120 together with the memory 130 (and associated modules), the models database 150 and the augmented database 155 in FIG. 1, for example. In block S211, a models database is populated by one or more pre-trained models. The models generally enable determination of what physical activities and postures correspond to various data collected by a sensor. As discussed above, the models are pre-trained with training data that is collected from the body of a training subject (which may also be subject ultimately monitored and evaluated) at a location on the training subject's body that corresponds to the primary location. That is, the training location (and the primary location) may be about the chest of the training subject since raw data acquired at the chest tends to provide very accurate determinations of physical activities and postures, because of the availability of additional data not available at other locations and because of the substantial unity between movement and positon of the chest and movement and position of the body at large. Other locations are contemplated. The pre-trained models may be stored and provided by models database 150 to determine the physical activities or postures from the raw data captured by the wearable sensor 110.

In block S212, raw data is collected at the sensor attached to the subject indicating characteristics of the body of the subject and/or ambient conditions. The raw data may include acceleration, physical movement, body position, heart rate, temperature, atmospheric pressure, and/or heart and lung sounds, for example. In block S213, a physical location of the sensor on the body, as well as changes to the physical location of the sensor, may be determined based at least in part on the raw data collected at the sensor in block S212, model trained for sensor localization, as well as the data trends and/or changes in data trends derived from the raw data. For example, detection of heart and/or lung sounds and detection of small rhythmic movements consistent with breathing indicate that the sensor worn on the subject's chest, whereas the absence of heart and/or lung sounds and the detection of large irregular movements consistent with the motion of an arm indicate that the sensor worn on the subject's wrist.

In block S214, it is determined whether the physical location of the sensor matches the primary location on the body of the subject. The primary location corresponds to a location on the body at which training data are collected for training the pre-trained models, e.g., which have been stored in the models database 150. Any other location on the subject's body at which the sensor may be located would be considered a secondary location for purposes of the discussion herein.

When it is determined that the physical location of the sensor matches the primary location (block S214: Yes), at least one of physical activity and posture of the subject is determined in block S215 using the raw data collected at the sensor, in accordance with a model selected from among the pre-trained models and retrieved from the model database. In other words, there is no need to map the raw data to any other location in order to perform data analysis identify the physical activities and postures of the subject.

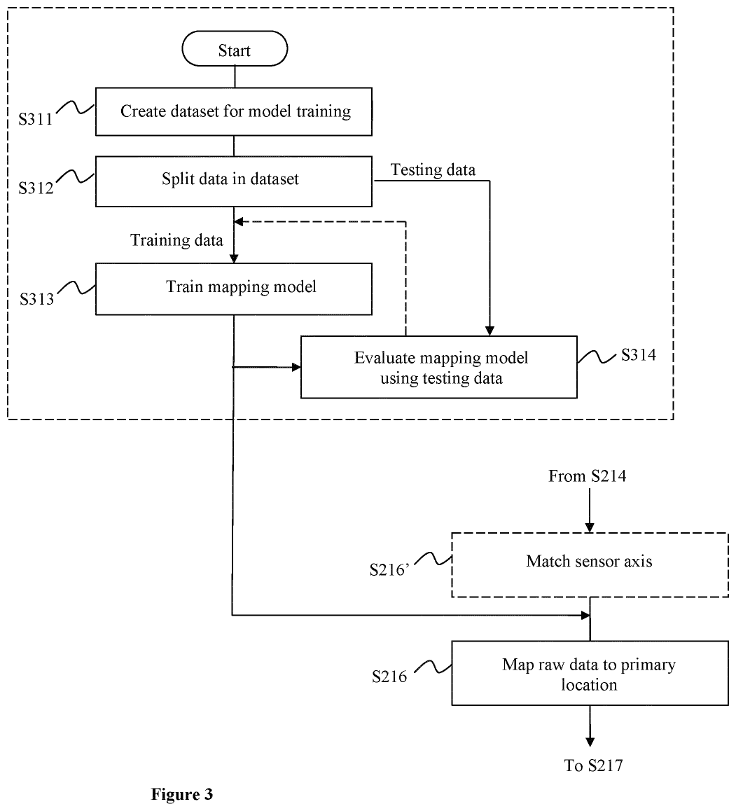

When it is determined that the physical location of the sensor does not match the primary location (block S214: No), the raw data is mapped from the actual physical location (which is a secondary location) of the sensor to the primary location in block S216 to provide mapped data. The mapping adjusts the raw data to account for differences in location between the secondary location, at which the sensor is actually positioned, and the primary location, for which the pre-trained models (including the selected model) are based. The mapping may be accomplished using a machine-learning based algorithm, an example of which is described below with reference to FIG. 3. In block S217, at least one of physical activity and posture of the subject is determined using the mapped data, in accordance with the selected model retrieved from the model database. That is, the mapped data is treated as if it were raw data collected by a sensor at the primary location, using the same selected model as used in block S215. Also, optionally, the mapped data may be recorded in an augmented database in block S218, and the selected model may be retrained using the mapped data recorded in the augmented database and the training data in block S219.

FIG. 3 illustrates a simplified flow diagram showing a process for mapping raw data from an actual physical location to a primary location of a sensor using a machine-learning based algorithm, in accordance with a representative embodiment, which may be executed by the various systems described herein. That is, FIG. 3 shows an example of implementing block S216 in FIG. 2.

Referring to FIG. 3, in block S311, a dataset is created for training a mapping model to map raw data from one (secondary) location on a subject's body to a primary location on the subject's body, the primary location being the location for which the one or more pre-trained models for determining physical activity and/or posture have been trained. The dataset may be created by simultaneously recording raw data at the secondary location and the primary location of the subject. Notably, the dataset may be recorded beforehand with different subjects. The present teachings also contemplate collecting data from the same subject and train a subject-specific model.

The dataset is split in block S312 into training data and testing data, where the training data is used to train a mapping model in block S313 and testing data is used to evaluate the trained mapping model in block S314. The mapping model is initially learned and obtained from training data in the training phase. The mapping model includes a machine-learning based algorithm, in that the trained mapping model is output from block S313 and evaluated in block S314 using the testing data. An output of block S314, indicating the performance of the mapping model based on the evaluation, might be used by block S313 for optimizing training of the mapping model, indicated by a dashed line, thereby improving performance of the mapping model.

The trained mapping model output from block S313 is also provided to block S216 in FIG. 2, for example, to enable mapping of the raw data from the actual physical location of the sensor to the primary location, when it has been determined in block S214 that the actual physical location of the sensor does not match the primary location. Also, FIG. 3 shows an illustrative embodiment that includes an additional block S216′, which precedes block S216, in which sensor axes of the sensor at the actual (secondary) physical location and a reference sensor at the primary location are matched. In this case, a reference frame of the raw data collected at the sensor is aligned with a reference frame of the training data used for developing the mapping model, for example, in order to minimize impact of sensor rotations and misalignment in sensors' coordinate frame. The mapped data is provided from block S216 to block S217 for determining at least one of physical activity and posture of the subject, as discussed above.

Referring again to FIG. 2, in block S220, the determined at least one of physical activity and posture of the subject are displayed on a display accessible to the sensor. For example, the sensor may include an integrated display one which the physical activity and/or posture are displayed. Also, the sensor may include a wireless interface, such as Bluetooth or Wi-Fi, which enables connection of the sensor to a remote computer, such as a PC or workstation. In this case, the physical activity and/or posture may be displayed on the remote computer. As discussed above, determining the physical activity and/or posture may performed by a processor included in the sensor, along with determining where on the body the sensor is located (block S214) and mapping the physical location of the sensor to the primary location, if needed (block S216). In this case, the physical activity and/or posture may be provided directly to the sensor display for viewing, or transmitted via wireless network connections to remote computer for additional viewing. In an alternative embodiment, the raw data may be transmitted via wireless network connections to the remote computer for processing, in which case determining the physical activity and/or posture may performed by the remote computer, along with determining where on the body the sensor is located (block S214) and mapping the physical location of the sensor to the primary location, if needed (block S216). In this case, the physical activity and/or posture may be provided directly to the remote computer display for viewing, or transmitted via wireless network connections back to the sensor for viewing on the sensor display.

Accordingly, by mapping raw data collected by a sensor at a secondary location to a primary location, the quality of the (mapped) data used for modeling and the quality of the corresponding modeling results is improved over use of only the secondary location (although the best results are still based on the raw data being collected at the same site on which the pre-trained model is based). Table 1 below compares the accuracies of results when (a) the raw data is collected at the same, most accurate location (chest) on which the selected model for determining physical activity and/or posture is based, (b) the raw data is collected at the same, less desirable location (wrist) on which the selected model for determining physical activity and/or posture is based, and (c) the raw data is collected at the less desirable location (wrist) and mapped to the better location (chest) on which the selected model for determining physical activity and/or posture is based. With regard to (c), an LSTM regression model to map the left wrist raw sensor data to chest sensor data. We could achieve low normalized root mean squared of 0.12±0.02. Table below demonstrates the average accuracy, balanced accuracy (average of specificity and sensitivity), and F1-score of estimating lying posture for held out test datasets. In the table we compare performance of lying posture estimation in three different scenarios:

TABLE 1

| | | | Balanced | |
|---|---|---|---|---|
| | Pre-Trained | Accuracy | Accuracy | |
| Sensor Data | Model | (%) | (%) | F1-Score |
| Chest | Chest Model | 94.6 ± 3.1 | 92.2 ± 3.7 | 92.2 ± 4.0 |
| Wrist | Wrist Model | 69.5 ± 9.3 | 65.3 ± 11.2 | 67.1 ± 13.4 |
| Wrist mapped to Chest | Chest Model | 75.2 ± 7.4 | 75.0 ± 6.3 | 74.8 ± 7.0 |

Referring to Table 1, the scenario in which the sensor data is collected at the chest and the pre-trained model is based on the chest location is the best scenario. This is because a chest pre-trained model is very accurate, and the raw data is collected by a sensor mounted on the chest provides the most accurate data for that model. This entry is the upper-bound to the accuracy that can be achieved. Where the raw data is collected from the wrist, and the pre-trained model is based on the wrist, baseline accuracy is provided. When the sensor data collected at the wrist is mapped to the chest, the regression LSTM-based model is trained on the wrist and chest training datasets to be able to map the currently available data from wrist sensor to a chest sensor data, so that the chest data is estimated from the available wrist sensor data. The, the accurate model trained on previous data from chest is applied to achieve higher accuracy than the baseline and close to the accuracy of the upper-bound. As shown in Table 1, the embodiment improves the baseline performance by about 7.5 percent, on average.

FIG. 4 illustrates a general computer system, on which a method of evaluating a subject using a wearable sensor may be implemented, in accordance with a representative embodiment.

Referring to FIG. 4, computer system 400 can include a set of instructions that can be executed to cause the computer system 400 to perform any one or more of the methods or computer-based functions disclosed herein. The computer system 400 may operate as a standalone device or may be connected, for example, using a network 401, to other computer systems or peripheral devices.

In a networked deployment, the computer system 400 may operate in the capacity of a server or as a client user computer in a server-client user network environment, or as a peer computer system in a peer-to-peer (or distributed) network environment. The computer system 400 can also be implemented as or incorporated into various devices, such as a stationary computer, a mobile computer, a personal computer (PC), a laptop computer, a tablet computer, a wireless smart phone, a personal digital assistant (PDA), or any other machine capable of executing a set of instructions (sequential or otherwise) that specify actions to be taken by that machine. The computer system 400 may be incorporated as or in a device that in turn is in an integrated system that includes additional devices. In an embodiment, the computer system 400 can be implemented using electronic devices that provide voice, video or data communication. Further, while the computer system 400 is illustrated in the singular, the term “system” shall also be taken to include any collection of systems or sub-systems that individually or jointly execute a set, or multiple sets, of instructions to perform one or more computer functions.

As illustrated in FIG. 4, the computer system 400 includes a processor 410, which is tangible and non-transitory, and is representative of one or more processors. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A processor is an article of manufacture and/or a machine component. The processor 410 for the computer system 400 is configured to execute software instructions to perform functions as described in the various embodiments herein. The processor 410 may be a general-purpose processor or may be part of an application specific integrated circuit (ASIC). The processor 410 may also be (or include) a microprocessor, a microcomputer, a processor chip, a controller, a microcontroller, a digital signal processor (DSP), a state machine, or a programmable logic device. The processor 410 may also be (or include) a logical circuit, including a programmable gate array (PGA) such as a field programmable gate array (FPGA), or another type of circuit that includes discrete gate and/or transistor logic. The processor 410 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 400 may include a main memory 420 and/or a static memory 430, where the memories may communicate with each other via a bus 408. Memories described herein are tangible storage mediums that can store data and executable instructions and are non-transitory during the time instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A memory described herein is an article of manufacture and/or machine component. Memories described herein are computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, Blu-ray disk, or any other form of storage medium known in the art. Memories may be volatile or non-volatile, secure and/or encrypted, unsecure and/or unencrypted.

As shown, the computer system 400 may further include a video display unit 450, such as a liquid crystal display (LCD), an organic light emitting diode (OLED), a flat panel display, a solid-state display, or a cathode ray tube (CRT). Additionally, the computer system 400 may include an input device 460, such as a keyboard/virtual keyboard or touch-sensitive input screen or speech input with speech recognition, and a cursor control device 470, such as a mouse or touch-sensitive input screen or pad. The computer system 400 can also include a disk drive unit 480, a signal generation device 490, such as a speaker or remote control, and a network interface device 440.

In an embodiment, as depicted in FIG. 4, the disk drive unit 480 may include a computer-readable medium 482 in which one or more sets of instructions 484, e.g. software, can be embedded. Sets of instructions 484 can be read from the computer-readable medium 482. Further, the instructions 484, when executed by a processor, can be used to perform one or more of the methods and processes as described herein. In an embodiment, the instructions 484 may reside completely, or at least partially, within the main memory 420, the static memory 430, and/or within the processor 410 during execution by the computer system 400.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 482 that includes instructions 484 or receives and executes instructions 484 responsive to a propagated signal; so that a device connected to a network 401 can communicate voice, video or data over the network 401. Further, the instructions 484 may be transmitted or received over the network 401 via the network interface device 440.

As described above, the present disclosure is not to be limited in terms of the particular embodiments described in this application, which are intended as illustrations of various aspects. Many modifications and variations can be made without departing from its spirit and scope, as may be apparent. Functionally equivalent methods and apparatuses within the scope of the disclosure, in addition to those enumerated herein, may be apparent from the foregoing representative descriptions. Such modifications and variations are intended to fall within the scope of the appended representative claims. The present disclosure is to be limited only by the terms of the appended representative claims, along with the full scope of equivalents to which such representative claims are entitled. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It may be understood by those within the art that terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It may be further understood by those within the art that if a specific number of an introduced claim recitation is intended, such an intent may be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It may be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” may be understood to include the possibilities of “A” or “B” or “A and B.”

The foregoing description, along with its associated embodiments, has been presented for purposes of illustration only. It is not exhaustive and does not limit the concepts disclosed herein to their precise form disclosed. Those skilled in the art may appreciate from the foregoing description that modifications and variations are possible in light of the above teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Accordingly, the present disclosure is not limited to the above-described embodiments, but instead is defined by the appended claims in light of their full scope of equivalents.

In the preceding, various preferred embodiments have been described with references to the accompanying drawings. It may, however, be evident that various modifications and changes may be made thereto, and additional embodiments may be implemented, without departing from the broader scope of the inventive concepts disclosed herein as set forth in the claims that follow. The specification and drawings are accordingly to be regarded as an illustrative rather than restrictive sense.

Although system and method of evaluating a subject using a wearable sensor have been described with reference to a number of illustrative embodiments, it is understood that the words that have been used are words of description and illustration, rather than words of limitation. Changes may be made within the purview of the appended claims, as presently stated and as amended, without departing from the scope and spirit of system and method of optimal sensor placement in its aspects. Although system and method of optimal sensor placement has been described with reference to particular means, materials and embodiments, system and method of optimal sensor placement is not intended to be limited to the particulars disclosed; rather system and method of evaluating a subject using a wearable sensor extend to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A method of evaluating a subject using a wearable sensor on a body of the subject, the method comprising:
collecting raw data at the sensor indicating characteristics associated with the body;
determining a physical location of the sensor on the body of the subject;
determining whether the physical location of the sensor matches a primary location on the body of the subject, the primary location corresponding to a location at which training data are collected for training pre-trained models stored in a model database;
when the physical location of the sensor matches the primary location, determining at least one of physical activity and posture of the subject, using the raw data collected at the sensor, in accordance with a model selected from among the pre-trained models and retrieved from the model database; and
when the physical location of the source sensor does not match the primary location:
mapping the raw data from the physical location to the primary location using a machine-learning based algorithm to provide mapped data; and
determining at least one of physical activity and posture of the subject, using the mapped data, in accordance with the selected model retrieved from the model database; and
displaying the determined at least one of physical activity and posture of the subject on a display accessible to the sensor.

2. The method of claim 1, further comprising:
recording the mapped data in an augmented database; and
retraining the selected model using the mapped data recorded in the augmented database and the training data.

3. The method of claim 1, wherein mapping the raw data from the physical location to the primary location comprises mapping a source time series from the sensor to a target time series of the mapped data using a neural network.

4. The method of claim 3, wherein the neural network comprises a recurrent neural network with long short-term memory (LSTM).

5. The method of claim 1, wherein determining the physical location of the sensor comprises receiving acceleration data from an accelerometer or a gyroscope on the sensor indicating movement of the sensor relative to the body of the subject.

6. The method of claim 1, wherein determining the physical location of the sensor comprises receiving audio data from a microphone on the sensor indicating proximity to heart or lungs of the subject.

7. The method of claim 1, wherein the physical location is on a wrist of the subject, and the primary location is on a chest of the subject.

8. The method of claim 1, further comprising:
determining whether the physical location of the sensor has changed to a new physical location; and
when the physical location of the sensor has changed, determining whether the new physical location matches the primary location.

9. The method of claim 4, wherein mapping the raw data from the physical location to the primary location using the machine-learning based algorithm comprises optimizing a number of LSTM layers and fully connected layers and regression layers.

10. The method of claim 1, wherein the characteristics associated with the body comprise at least one of acceleration, physical movement, body position, heart rate, temperature, atmospheric pressure, and heart and lung sounds.

11. A sensor device, wearable on a body of a subject at a primary location or at other locations, for determining at least one of physical activity and posture of the subject, the device comprising:
a database that stores at least one pre-trained model previously trained using training data recorded from the primary location;
a memory that stores executable instructions comprising a sensor localization module, a physical activity and posture recognition module, and a sensor data mapping module; and
a processor configured to execute the instructions retrieved from the memory, wherein the instructions, when executed, cause the processor to:
collect raw data indicating characteristics associated with the body in accordance with user instructions;
determine a location of the sensor device on the body of the subject in accordance with the sensor localization module;
determine whether the determined location matches the primary location in accordance with the sensor localization module;
when the determined location matches the primary location, determine at least one of physical activity and posture of the subject, using the collected raw data and a pre-trained model selected from the at least one model stored in the database, in accordance with the physical activity and posture recognition module; and
when the determined location does not match the primary location, map the raw data from the determined location to the primary location to provide mapped data, in accordance with the sensor data mapping module, and determine at least one of physical activity and posture of the subject, using the mapped data, and a pre-trained model selected from the at least one model stored in the database, in accordance with the physical activity and posture recognition module; and
a display configured to display the at least one of physical activity and posture of the subject determined in accordance with the physical activity and posture recognition module.

12. The sensor device of claim 11, wherein the processor maps the raw data from the determined location to the primary location using a machine-learning based algorithm.

13. The sensor device of claim 11, further comprising:
an augmented database that stores the mapped data, wherein the at least one model stored in the database is retrained using the mapped data stored in the augmented database.

14. The sensor device of claim 11, wherein the processor maps the raw data from the determined location to the primary location by mapping a source time series from the sensor device to a target time series of the mapped data using a recurrent neural network with long short-term memory (LSTM).

15. The sensor device of claim 11, wherein the processor determines the determined location of the sensor device on the body by receiving acceleration data from an accelerometer or a gyroscope indicating movement of the sensor device relative to the body of the subject.

16. The sensor device of claim 11, wherein the processor determines the determined the location of the sensor device by receiving audio data from a microphone indicating proximity to heart or lungs of the subject.

17. The sensor device of claim 11, wherein the primary location is on a chest of the subject.

18. The sensor device of claim 12, wherein mapping the machine-learning based algorithm comprises a recurrent neural network with long short-term memory (LSTM).

19. The sensor device of claim 11, wherein the characteristics associated with the body comprise at least one of acceleration, physical movement, body position, heart rate, temperature, atmospheric pressure, and heart and lung sounds.

20. A non-transitory computer readable medium for enabling evaluation of a subject using a wearable sensor on a body of the subject, the computer readable medium storing instructions that, when executed by a processor, cause the processor to perform a method comprising:
determining a physical location of the sensor on the body of the subject;
determining whether the physical location of the sensor matches a primary location on the body of the subject, the primary location corresponding to a location at which training data are collected for training pre-trained models stored in a model database;
when the physical location of the sensor matches the primary location, determining at least one of physical activity and posture of the subject, using raw data collected at the sensor, indicating characteristics associated with the body, in accordance with a model selected from among the pre-trained models and retrieved from the model database; and
when the physical location of the source sensor does not match the primary location:
mapping the raw data from the physical location to the primary location using a machine-learning based algorithm to provide mapped data; and
determining at least one of physical activity and posture of the subject, using the mapped data, in accordance with the selected model retrieved from the model database; and
displaying the determined at least one of physical activity and posture of the subject on a display accessible to the sensor.
